(19) United States
(12) Patent Application Publication
(10) Pub. No.: US 2006/0240431 A1
(43) Pub. Date: Oct. 26, 2006

(54) Oligonucletide guided analysis of gene expression

(76) Inventor: Guoliang Fu, Oxford (GB)

Correspondence Address: PATREA L. PABST;PABST PATENT GROUP LLP, 400 COLONY SQUARE, SUITE 1200, ATLANTA, GA, 30361, US

(21) Appl. No.: 10/537,737

(22) PCT Filed: Dec. 3, 2003
(86) PCT No.: PCT/GB03/05271

§ 371 Date: Jun. 7, 2006

(30) Foreign Application Priority Data
Dec. 7, 2002 (GB) 0228614.4

(51) Int. Cl. C12Q 1/68; C07H 21/04

(52) U.S. Cl. 435/6; 536/24.3

(57) ABSTRACT

The present invention relate to methods and compositions for simultaneously analyzing multiple different polynucleotides of a nucleic acid sample. The subject methods and compositions may also be applied to analyze or identify single polynucleotide; however, the subject methods and compositions are particularly useful for analyzing large diverse populations of polynucleotides. Methods of the invention involve hybridizing guide oligonucleotides to target polynucleotides for analysis, subsequently digesting double-stranded or partially double-stranded guide oligonucleotide intermediates, and isolating and analyzing digested part. The guide oligonucleotide is marked in identifier sequence and constant region so as to facilitate the simultaneous testing of multiple target polynucleotides. The identity or expression of a particular polynucleotide of interest may be ascertained by producing and quantifying a short identifier sequence derived from combining guide oligonucleotides and target polynucleotides.

### FIELD OF THE INVENTION

The invention relates generally to methods and compositions for quantitative analysis of nucleic acids, and more particularly, to methods and compositions for analyzing sequence tags derived from combining guide oligonucleotides and target polynucleotides.

### BACKGROUND

The desire to decode the human genome and to understand the genetic basis of disease and a host of other physiological states associated differential gene expression has been a key driving force in the development of improved methods for analyzing nucleic acids. The human genome is estimated to contain over 30,000 genes, about 15-30% of which are active in any given tissue. Such large numbers of expressed genes make it difficult to track changes in expression patterns by available techniques, such as with hybridization of gene products to microarrays, direct sequence analysis, or the like. More commonly, expression patterns are initially analyzed by lower resolution techniques, such as differential display, indexing, subtraction hybridization, or one of the numerous DNA fingerprinting techniques. Higher resolution analysis is then frequently carried out on subsets of cDNA clones identified by the application of such techniques.

Recently, two techniques have been implemented that attempt to provide direct sequence information for analyzing patterns of gene expression. One involves the use of microarrays of oligonucleotides or polynucleotides for capturing complementary polynucleotides from expressed genes, e.g. Schena et al, Science, 270: 467-469 (1995); DeRisi et al, Science, 278: 680-686 (1997); Chee et al, Science, 274: 610-614 (1996); and the other involves the excision and concatenation of short sequence tags from cDNAs, followed by conventional sequencing of the concatenated tags, i.e. serial analysis of gene expression (SAGE), e.g. Velculescu et al, Science, 270: 484-486 (1995); Zhang et al, Science, 276: 1268-1272 (1997); Velculescu et al, Cell, 88: 243-251 (1997). Both techniques have shown promise as potentially robust systems for analyzing gene expression; however, there are still technical issues that need to be addressed for both approaches. For example, in microarray systems, genes to be monitored must be known and isolated beforehand, and with respect to current generation microarrays, the systems lack the complexity to provide a comprehensive analysis of mammalian gene expression, they are not readily re-usable, and they require expensive specialized data collection and analysis systems, although these of course may be used repeatedly. In SAGE systems, although no special instrumentation is necessary and an extensive installed base of DNA sequencers may be used, the selection of type IIs tag-generating enzymes is limited, and the length (ten nucleotides) of the sequence tag in current protocols severely limits the number of cDNAs that can be uniquely labeled. One limitation of SAGE may be that a large portion of cost and time are spent on sequencing non-informative sequence tags e.g. those are derived from high abundant house keeping genes. In addition, the SAGE is limited to analyze only a portion of the expressed genes as the form of mRNA

It is clear from the above that there is a need for a technique to quickly and inexpensively analyze gene expression, not only the mRNA, but all other non-mRNA gene expression. The availability of such techniques would find immediate application in medical and scientific research, drug discovery, and genetic analysis in a host of applied fields.

### SUMMARY OF THE INVENTION

The present invention relate to methods and compositions for simultaneously analyzing multiple different polynucleotides of a nucleic acid sample. The subject methods and compositions may also be applied to analyze or identify single polynucleotides; however, the subject methods and compositions are particularly useful for analyzing large diverse populations of polynucleotides. Most embodiments of the invention involve hybridizing guide oligonucleotides to total RNA, genomic DNA, or cDNA for analysis, subsequently digesting double-stranded or partially double-stranded guide oligonucleotide intermediates, and isolating and analyzing digested part. The guide oligonucleotide may be marked in identifier sequence region and constant region so as to facilitate the simultaneous testing of multiple polynucleotides for the presence of specific targets. The identity or expression of a particular polynucleotide of interest may be ascertained by producing and quantifying a short identifier sequence derived from combining guide oligonucleotides and target polynucleotides. Multiple identification sequences may be obtained in parallel, thereby permitting the rapid characterization of a large number of diverse polynucleotides.

A guide oligonucleotide is single-stranded or partially double-stranded nucleic acid, which comprises: target complementary region, constant region, identifier sequence, at least one restriction site. Said at least one restriction site comprises the first and second restriction sites which are different, wherein said second restriction site is adjacent to said constant region.

Said identifier sequence is specific for each said guide oligonucleotide and is located between the first and second restriction sites. Said constant region is located at the most 3′ or 5′ end of said guide oligonucleotide, wherein said constant region comprises sequence complementary or identical to an amplification primer sequence.

The guide oligonucleotide may further comprise 5′ or 3′ end label. Said end label may comprise biotin.

The identifier sequence and first restriction site may be part of the target complementary region. The identifier sequence and first restriction site may be not part of the target complementary region.

The guide oligonucleotide may further comprise additional enzyme acting site which supports digestion of target sequence strand hybridized to said target complementary region of said guide oligonucleotide. The additional enzyme acting site may comprise restriction site. The restriction site may comprise type IIS restriction site or nicking restriction site. The enzyme recognition sites of type IIS restriction site or nicking restriction site may be double-stranded by hybridization with helper primer. The nucleotides of the cleavage site of said restriction site on the target complementary region may be modified, whereby the modified nucleotides are resistant to cleavage. The modified nucleotides may comprise phosphorothioate linkages.

Said additional enzyme acting site may comprise RNase H digestion sites when the target is RNA. The target complementary region of said guide oligonucleotide may comprise chimeric RNA and DNA.

A set of guide oligonucleotides comprises multiple guide oligonucleotides each having a target specific target complementary region, a guide oligonucleotide specific identifier sequence, the same first restriction site, the same second restriction site, and the same constant region sequence.

A method of analyzing polynucleotides in a sample, said method comprising steps of: (a) hybridizing guide oligonucleotides or a set of guide oligonucleotides or more than one set of guide oligonucleotides to target polynucleotides, whereby target complementary regions of said guide oligonucleotides become double-stranded if the targets are present in the sample; (b) forming double-stranded or partially double-stranded guide oligonucleotide intermediates including double-stranded first restriction sites; (c) digesting said double-stranded or partially double-stranded guide oligonucleotides with first restriction enzyme on the first restriction site; and (d) analyzing the digested parts containing identifier sequences and constant regions.

In one embodiment, the first restriction sites and identifier sequences form part of the target complementary regions of the guide oligonucleotides, said step (b) is completed after said step (a).

In another embodiment, the target polynucleotides are RNA, said step (b) of forming double-stranded or partially double-stranded guide oligonucleotide intermediates comprises: partially digesting the target RNA strand of RNA/DNA hybrid by a nuclease, extending the 3′ end of digested strand on guide oligonucleotide templates by a DNA polymerase, whereby the downstream sequences 5′ to the target complementary region of the guide oligonucleotide including the first restriction site become double-stranded. Said nuclease may be RNase H.

In still another embodiment, the guide oligonucleotides comprise additional restriction sites, said step (b) of forming double-stranded or partially double-stranded guide oligonucleotide intermediates comprises: digesting target sequence strand by the restriction enzyme on restriction digestion sites of said additional restriction site, extending the 3′ end of the digested strand on guide oligonucleotide templates by a DNA polymerase, whereby the downstream sequences 5′ to the target complementary region of the guide oligonucleotide including the first restriction site become double-stranded.

In still another embodiment, the target complementary regions of said guide oligonucleotides hybridize to free 3′ ends of the target sequences, and said step (b) of forming double-stranded or partially double-stranded guide oligonucleotide intermediates comprises: extending said free 3′ ends of the target sequences by a nucleic acid polymerase using said guide oligonucleotides as templates, whereby the downstream sequences 5′ to the target complementary region of the guide oligonucleotide including the first restriction site become double-stranded.

In still another embodiment, said step (b) of forming double-stranded or partially double-stranded guide oligonucleotide intermediates comprises: trimming single-stranded target sequence 3′ to the target region hybridized to the guide oligonucleotide with an exonuclease activity, extending 3′ ends of the trimmed target sequences by a nucleic acid polymerase using said guide oligonucleotides as templates, whereby the downstream sequences 5′ to the target complementary region of the guide oligonucleotide including the first restriction site become double-stranded. In this embodiment, said guide oligonucleotide comprises at least one modified nucleotide or modified phosphodiester, linkage in at least an ultimate 3′ end position to resist exonuclease activity.

After said step (a) or step (b), the method may further comprise: capturing said polynucleotide or said oligonucleotide on a solid support through the end labels, and stringency washing.

After said step (c), the method may further comprise: isolating the digested parts containing identifier sequences and constant regions, wherein said digested parts are attached on the solid support or in supernatant.

In one embodiment, said step (d) of analyzing the digested parts containing identifier sequences and constant regions comprises: detecting said digested parts by mass spectrometry, electrophoresis or microarray.

In another embodiment, said step (d) of analyzing the digested parts containing identifier sequences and constant regions comprises: ligating said digested parts to each other by a nucleic acid ligase to produce at lease one joined identifier fragment, amplifying joined identifier fragments using primers that are complementary or identical to constant regions of the guide oligonucleotides, analyzing the amplified products. In a sub-embodiment, said analyzing the amplified products comprises determining the nucleotide sequence of said amplified products. In another sub-embodiment, said analyzing the amplified products comprises: digesting said amplified products with first and second restriction enzymes to release individual identifier sequences, detecting and quantifying said identifier sequences by a detection method. Said detection method may comprise mass spectrometry, electrophoresis or microarray. In still another sub-embodiment or a preferred sub-embodiment, said analyzing the amplified products comprises: digesting said amplified products with second restriction enzymes to release joined identifier sequences, ligating said joined identifier sequences to produce concatemers, determining the nucleotide sequence of identifier sequences in said concatemers. Said determining the nucleotide sequence of identifier sequences in said concatemers may comprise cloning, sequencing and counting the numbers of identifier sequences.

### BRIEF DESCRIPTION OF DRAWINGS

FIG. 1 is schematic diagram showing guide oligonucleotides. The functional regions of the guide oligonucleotide are indicated.

FIG. 2 is a schematic diagram of a method of analyzing complex polynucleotides in accordance with the methods of the invention.

FIG. 3 is a schematic diagram of a method of analyzing complex polynucleotides using guide oligonucleotides having their first restriction sites and identifier sequences forming part of target complementary regions.

FIG. 4 is a schematic diagram of analyzing biotinilated cDNA.

### DESCRIPTION OF THE PREFERRED EMBODIMENTS

The present invention relate to methods and compositions for simultaneously analyzing multiple different polynucleotides of a polynucleotide composition comprising multiple diverse polynucleotide sequences. The subject methods and compositions may also be applied to analyze or identify single polynucleotides; however, the subject methods and compositions are particularly useful for analyzing large diverse populations of polynucleotides. Most embodiments of the invention involve hybridizing guide oligonucleotides to RNA, genomic DNA, or cDNA for analysis, subsequently digesting double-stranded or partially double-stranded guide oligonucleotide intermediates, and isolating and analyzing digested part. The guide oligonucleotide may be marked in its identifier sequence and constant region so as to facilitate the simultaneous testing of multiple polynucleotides for the presence of particular targets. The identity or expression of a particular polynucleotide of interest may be ascertained by producing and quantifying a short identifier sequence derived from guide oligonucleotides. Multiple identification sequences may be obtained in parallel, thereby permitting the rapid characterization of a large number of diverse polynucleotides.

Analysis of polynucleotide populations in accordance with methods of the invention may be used to provide one or more of the following types of information: (1) the nucleotide sequence of one or more polynucleotides in a complex polynucleotide composition, or (2) the relative concentrations of one or more different polynucleotides in a complex polynucleotide composition. Analysis of large complex populations of polynucleotides by the subject methods may be used to produce sufficient information about a polynucleotide population that differences between polynucleotide populations may be ascertained.

Guide Oligonucleotide

Guide oligonucleotide is a linear single-stranded or partially double-stranded nucleic acid molecule, generally containing between 30 to 1000 nucleotides, preferably between about 40 to 300 nucleotides, and most preferably between about 50 to 150 nucleotides. Regions of guide oligonucleotides have specific functions making the guide oligonucleotide useful for embodiments of invention. A guide oligonucleotide generally comprises target complementary region, constant region, identifier sequence, at least one restriction site—usually there are two restriction sites termed as first and second restriction sites, with or without 5′ or 3 end label. A guide oligonucleotide may comprise additional enzyme acting sequence and helper primer.

1. Target Complementary Region

The target complementary region of a guide oligonucleotide is complementary or substantially complementary to a target region of interested target polynucleotide. The target region of interest chosen may be any desirable sequence, which may comprise SNP site, mutation sequence, methylation site, splicing site, restriction site, and any particular sequence of interest.

The target complementary region of a guide oligonucleotide can be any length that supports specific and stable hybridization between the guide oligonucleotide and the target sequence. For this purpose, a length of 9 to 60 nucleotides for target complementary region is preferred, with target complementary regions 15 to 40 nucleotides long being most preferred.

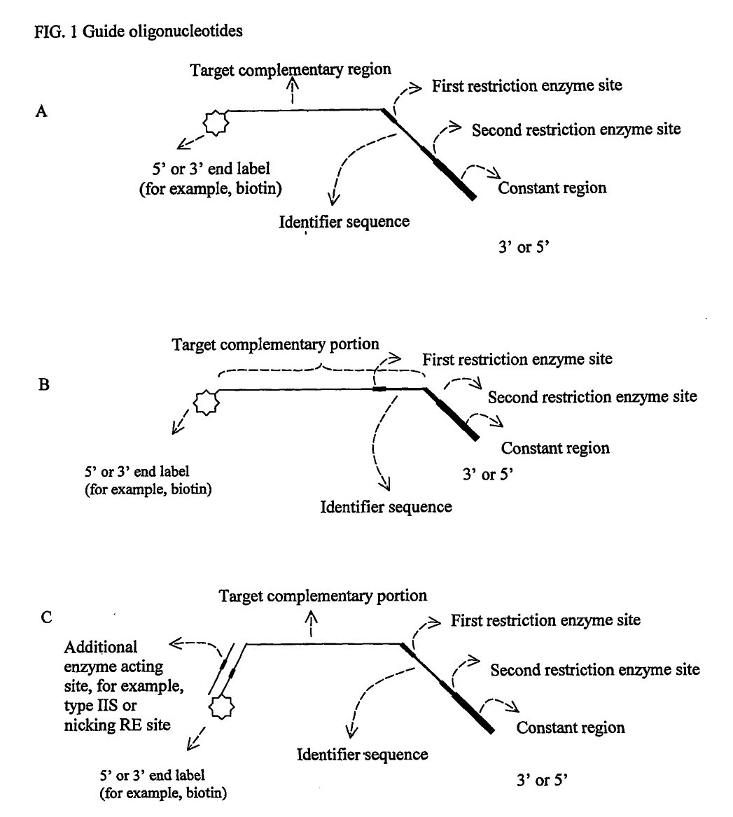

The target complementary region of the guide oligonucleotide becomes double-stranded after specific hybridization between the target sequence and the guide oligonucleotide. In one embodiment, the first restriction site and identifier sequence form part of target complementary region (FIG. 1B), upon hybridization of guide oligonucleotide to the target region of interest, the first restriction site become double-stranded and functional. In another embodiment, the target region that hybridizes to the target complementary region of the guide oligonucleotide is digested or nicked by digesting agents that act on the additional enzyme acting sequence of the guide oligonucleotide. The 3′ end of digested strand then is extended by a DNA polymerase using the guide oligonucleotide as templates, whereby the downstream first restriction site and other regions become double-stranded. In still another embodiment, the target complementary region hybridizes to free 3′ end(s) of the target sequence(s), which are extended by a DNA polymerase using the guide oligonucleotide as template, whereby the downstream first restriction site and other regions become double-stranded.

In further embodiments, the target sequence is RNA, upon hybridization to the target complementary region, the target RNA sequence in the hybrid RNA/DNA can be partially digested by RNase H digestion at various non-specific sites. It is preferred that some part (preferably the 3′ part) of the target complementary region can be made by RNA. Upon hybridization between target RNA sequence and the target complementary region, the RNA/RNA hybrid is resistant to digestion with RNase H. This is beneficial that the target RNA in the hybrid formed between target and guide oligonucleotide is not digested away so that partially digestion and extension can occur.

2. Constant Region

The constant region serves as priming site for amplification. In other words, the constant region is complementary or identical to primer sequence used for amplification. For this purpose, a length of 15 to 50 nucleotides for the constant region is preferred, and 18 to 35 nucleotides long are most preferred. The “constant region” is said to be constant because the constant regions in a set of guide oligonucleotides are functionally the same to each other with respect to their hybridization specificity to amplification primers as used in the methods of the invention. The constant region can have any desired sequence. In general, the sequence of the constant region can be chosen such that it is not significantly similar to any sequence in target polynucleotides.

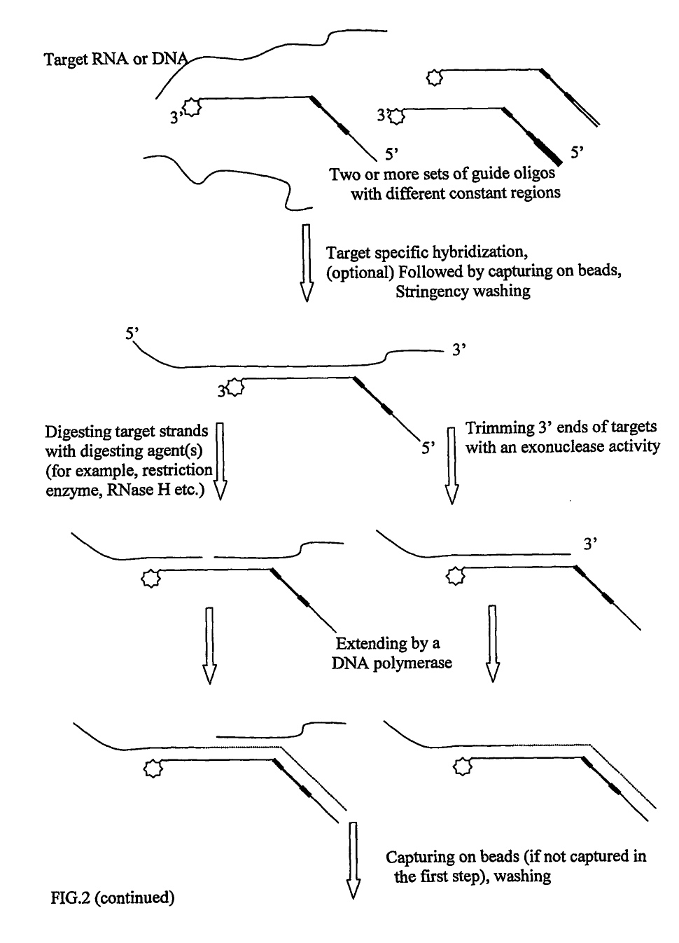

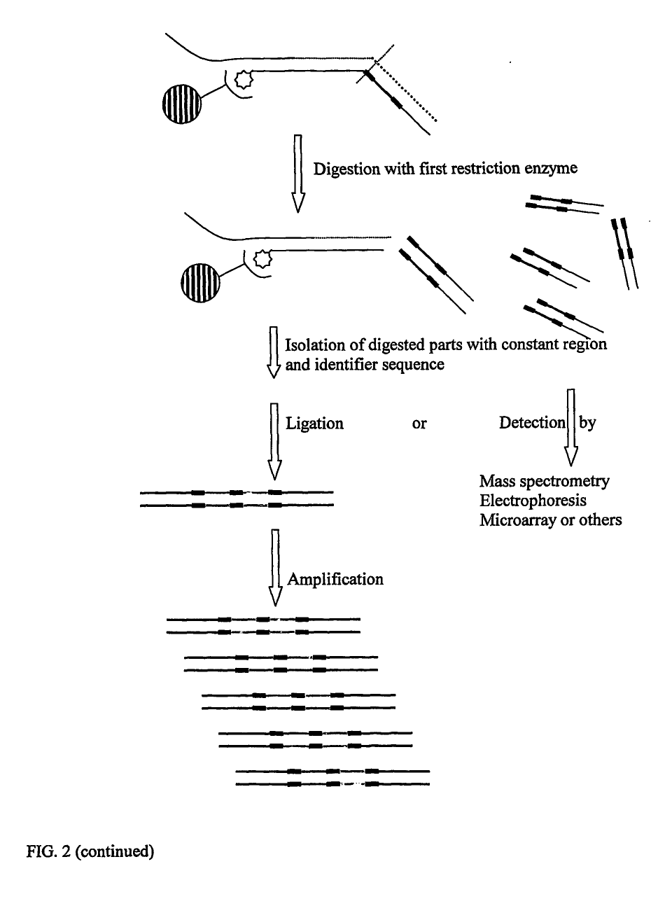

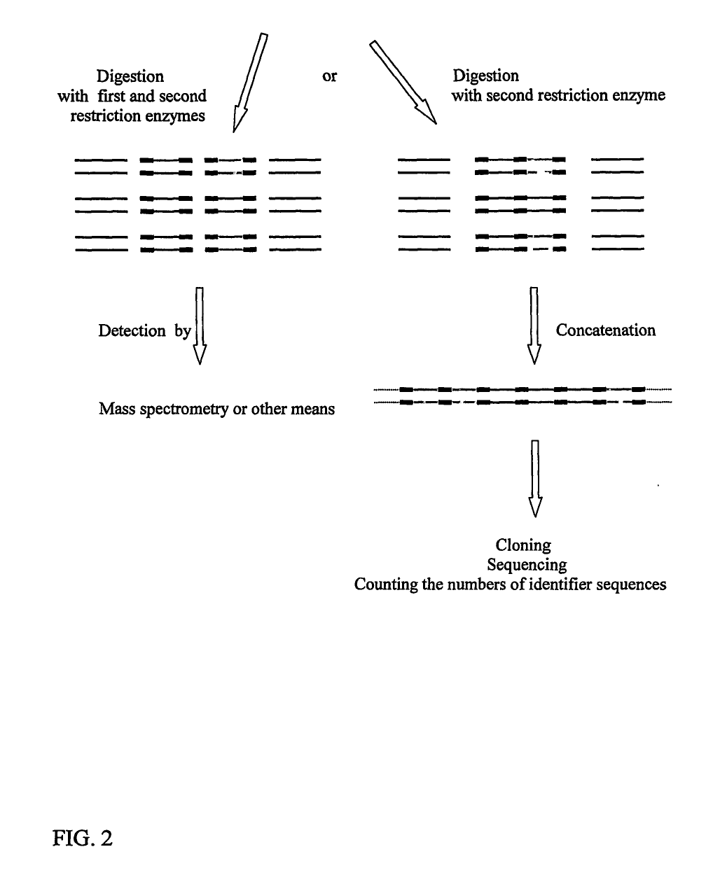

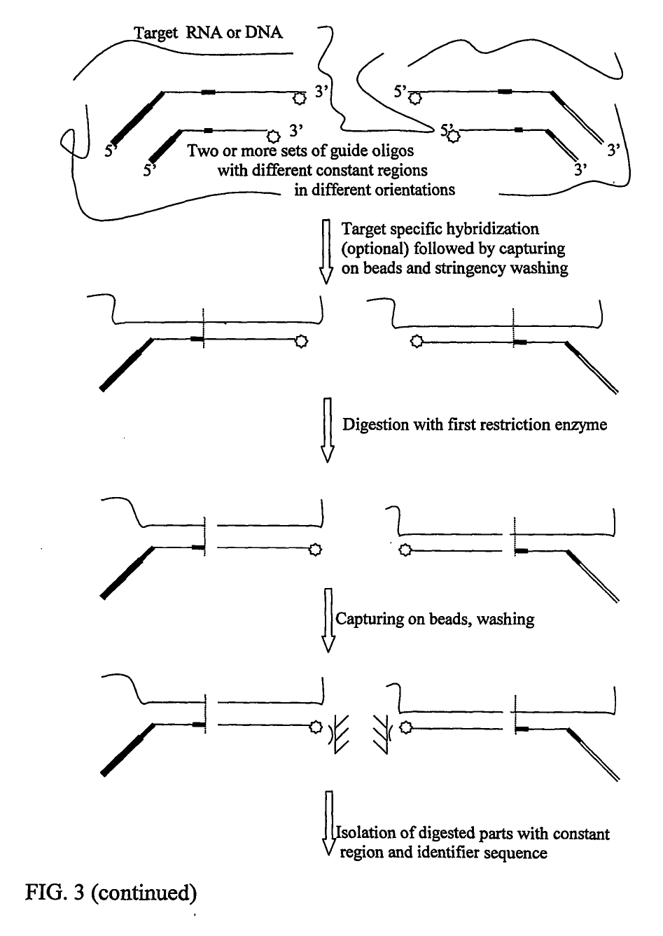

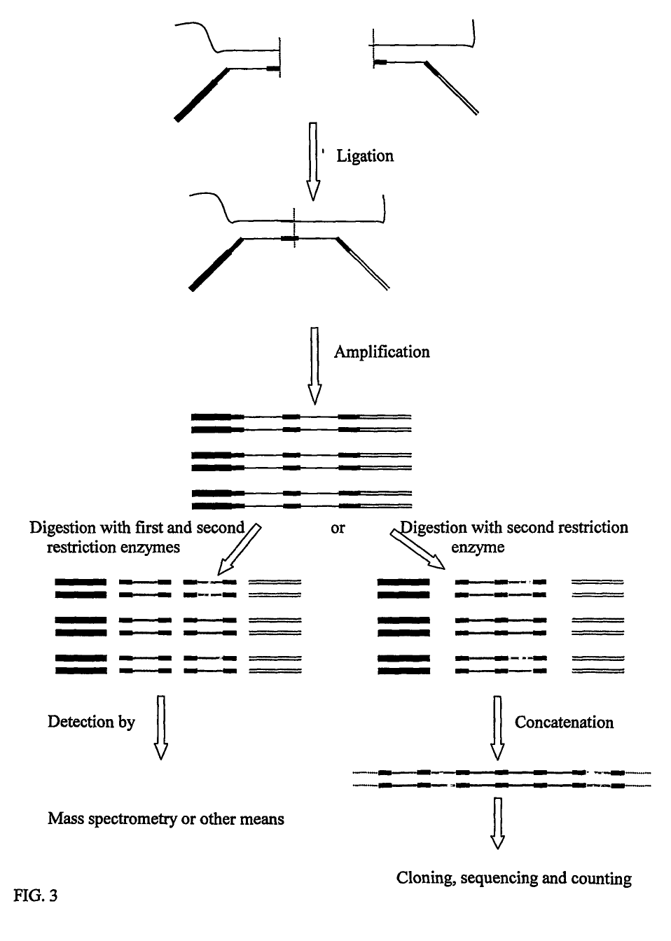

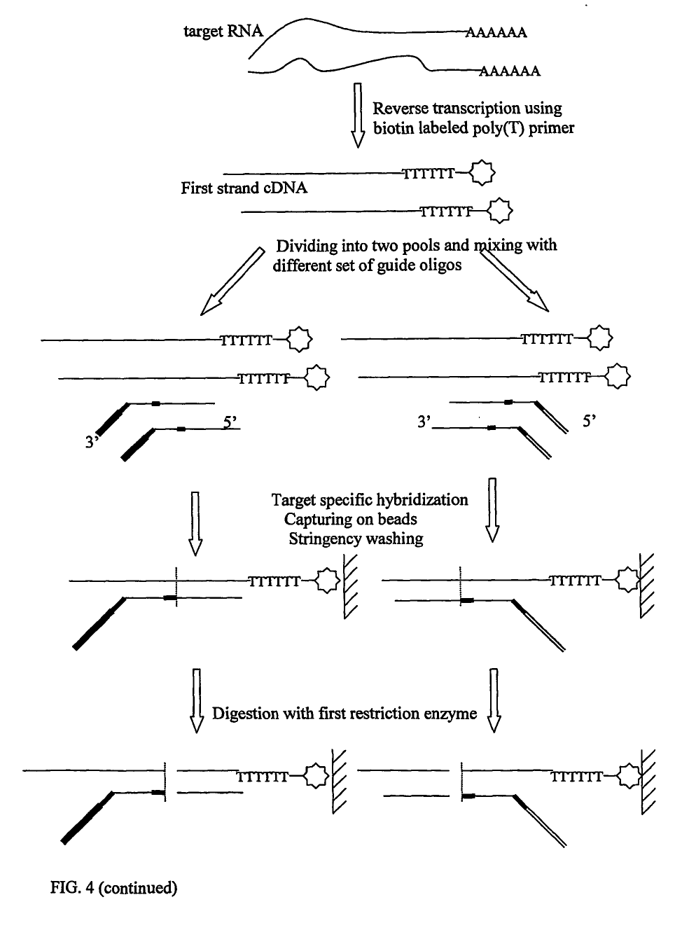

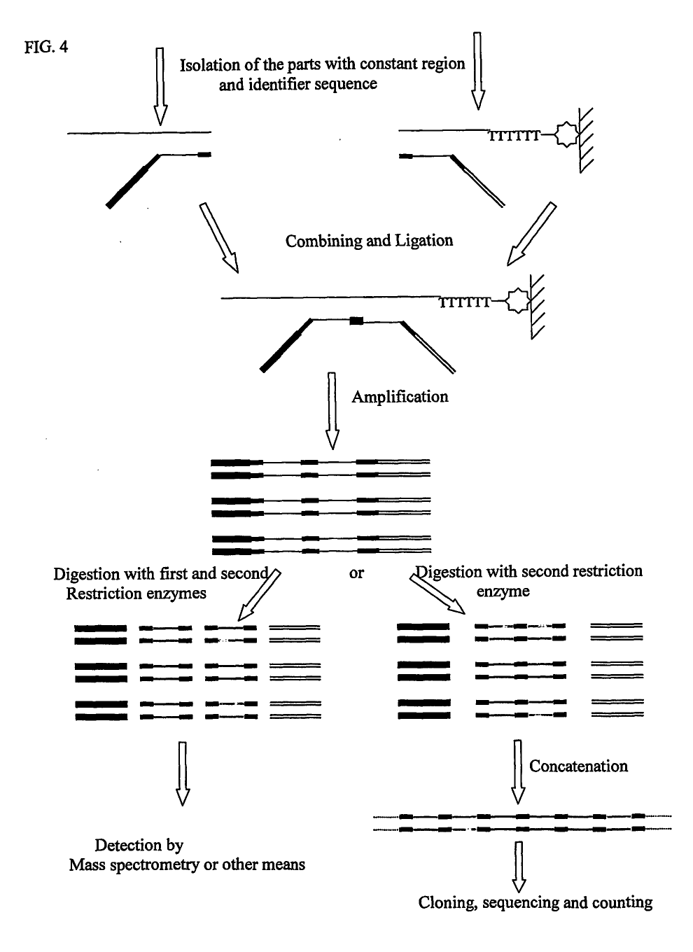

The constant region of a guide oligonucleotide is located at the most 3′ or 5′ end of the guide oligonucleotide. The selection of the relative orientation of the constant region with respect to the target complementary region in a given embodiment of the invention will vary in accordance with choice of which part of target polynucleotide is selected for analysis. In some embodiments of invention, a set or several sets of guide oligonucleotides have the same orientation of the constant regions, but the sequences of constant regions are different between different sets of oligos (FIG. 2). In other embodiments of invention, a set or several sets of guide oligonucleotides have the different orientations of the constant regions, as well as different sequences of constant regions between different sets of the guide oligonucleotide (FIG. 3 and FIG. 4).

The term “a set of guide oligonucleotides” as used herein refers to a plurality of different guide oligonucleotides used in conjunction with each other, wherein each guide oligonucleotide in the set has a functionally identical constant region, e.g., all of the constant regions are identical or have essentially the same properties for hybridization with an amplification primer, and each guide oligonucleotide in the set has a target complementary region with similar properties for hybridization to their target sequences, e.g., the target complementary region sequences of all of the guide oligonucleotides in the set have a similar annealing temperature. Each guide oligonucletide in a set of guide oligonucleotides may have the same first restriction site and the same second restriction site. The constant region sequences between different sets of guide oligonucleotodes are preferably different, whereas the first restriction sites and second restriction sites may be the same or different between different sets of guide oligonucleotodes.

3. Identifier Sequence

Identifier sequence is located between first and second restriction sites. Identifier sequence can comprise any sequence of any length that is unique to a guide oligonucleotide. The identifier sequence serves as a role to distinguish individual guide oligonucleotides. For this purpose, a length of 4 to 30 nucleotides for the identifier sequence is preferred, and 5 to 20 nucleotides long are most preferred. The identifier sequence can have any desired sequence. In some embodiments of the invention, the identifier sequence and first restriction site are contiguous to and form part of target complementary region. In other embodiments of the invention, the identifier sequence can be randomly chosen, and may not contain any significant similar sequence to target polynucleotides. All identifier sequences of the guide oligonucleotides in a set are not needed to be the same length. The identity of an identifier sequence may be determined by both its length and the sequence.

An identifier sequence is specifically associated with a given guide oligonucleotide, which is specifically associated with a target sequence, therefore the identifier sequence functions as a signature for the guide oligonucleotide and its associated target. In some embodiments, the method of the invention is used for determining the abundance and nature of transcripts corresponding to expressed genes. The method of the invention is based on the identification of and characterization of identified sequences derived from guide oligonucleotides hybridized to targets. The identifier sequences are markers for genes which are expressed in a cell, a tissue, or an extract, for example.

4. First and Second Restriction Enzyme Sites

Any restriction enzyme sites can be used as first and second restriction enzyme sites. In general, four base and six base cutters can be used, and four base cutters are preferred for the first restriction site. The first and second restriction sites are different. In some embodiments of the invention, the identifier sequence and first restriction site are contiguous to and form part of target complementary region. In other words, the target complementary region, first restriction enzyme and identifier sequence act as a whole to hybridize a target sequence. The first restriction site is located within the target complementary region or on either side 5′ or 3′ of the target complementary region. The second restriction site is adjacent to the constant region.

5. End Labels and Nucleotide Modifications

In certain embodiments, guide oligonucleotide can include one or more moieties incorporated into 5′ or 3′ terminus or internally of guide oligonucleotide that allow for the affinity separation of products derived from guide oligonucleotide associated with the label from unassociated parts. Preferred capture moieties are those that can interact specifically with a cognate ligand. For example, capture moiety can include biotin, digoxigenin etc. Other examples of capture groups include ligands, receptors, antibodies, haptens, enzymes, chemical groups recognizable by antibodies or aptamers. The capture moieties can be immobilized on any desired substrate. Examples of desired substrates include, e.g., particles, beads, magnetic beads, optically trapped beads, microtiter plates, glass slides, papers, test strips, gels, other matrices, nitrocellulose, nylon. For example, when the capture moiety is biotin, the substrate can include streptavidin.

In some embodiments, it may be desirable to modify the nucleotides or phosphodiester linkages in one or more positions of the guide oligonucleotide. For example, it may be advantageous to modify at least the 3′ portion of the guide oligonucleotide. Such a modification prevents the exonuclease activity from digesting any portion of the guide oligonucleotide. It is preferred that at least the ultimate and penultimate nucleotides or phosphodiester linkages be modified. In another example, the nucleotides of the cleavage site of the additional restriction site on the target complementary region may be modified. Such a modification prevents the endonuclease activity from digesting endonuclease digestion site of the guide oligonucleotide. One such modification comprises a phosphorothioate compound which, once incorporated inhibits 3′ exonucleolytic activity and endonuclease activity on the guide oligonucleotide. It will be understood by those skilled in the art that other modifications of the guide oligonucleotide, capable of blocking the exonuclease activity can be used to achieve the desired enzyme inhibition.

Extension of a guide oligonucleotide by a polymerase may be blocked by a blocking group at its 3′ end. The blockage of 3′ end of guide oligonucleotide can be achieved by any means known in the art. Blocking groups are chemical moieties which can be added to a nucleic acid to inhibit nucleic acid polymerization catalyzed by a nucleic acid polymerase. Blocking groups are typically located at the terminal 3′ end of guide oligonucleotide which is made up of nucleotides or derivatives thereof. By attaching a blocking group to a terminal 3′ OH, the 3′ OH group is no longer available to accept a nucleoside triphosphate in a polymerization reaction. Numerous different groups can be added to block the 3′ end of a probe sequence. Examples of such groups include alkyl groups, non-nucleotide linkers, phosphorothioate, alkane-diol residues, peptide nucleic acid, and nucleotide derivatives lacking a 3′ OH (e.g., cordycepin).

6. Additional Enzyme Acting Sequence

The guide oligonucleotide may further comprise additional enzyme acting sequence which supports digesting or nicking target sequence strand hybridized to the target complementary region of the guide oligonucleotide.

The additional enzyme acting sequence may comprise restriction site. The additional restriction site may be located within the target complementary region or on either side 3′ or 5′ to the target complementary region of the guide oligonucleotide. The nucleotides of the cleavage site of the additional restriction site on the target complementary region may be modified, whereby the modified nucleotides are resistant to cleavage. For example, it may be advantageous to modify restriction cleavage site of the guide oligonucleotide. Such a modification prevents the endonuclease activity from digesting endonuclease digestion site of the guide oligonucleotide. It is preferred that the nucleotides or phosphodiester linkages of endonuclease digestion site are modified. One such modification comprises a phosphorothioate compound which, once incorporated inhibits endonucleolytic activity on the guide oligonucleotide. It will be understood by those skilled in the art that other modifications of the guide oligonucleotide, capable of blocking the endonuclease activity can be used to achieve the desired enzyme inhibition.

The additional restriction site may be a type IIS restriction site or a nicking restriction site. The recognition sequences of type IIS restriction site or nicking restriction site may be double-stranded which are formed by hybridizing to helper primer. In one embodiment, a guide oligonucleotide comprises a type IIS restriction enzyme site as an additional enzyme acting sequence which is located 5′ to the target complementary region and of which the recognition sequence is double stranded by hybridizing to a helper primer (FIG. 1C). Because the type IIS enzymes cut several bases away from its restriction recognition sequence, the cleavage site can be or is preferred to be located on the target complementary region of the guide oligonucleotide. The nucleotide(s) on the cleavage site of the target complementary region may be modified to block cleavage of the guide oligonucleotide. To be functional, the type IIS restriction site of the guide oligonucleotide must be converted to double-stranded form for both its recognition sequence and cleavage site. The hybridization between the target and the guide oligonucleotide creates double-stranded cleavage site for type IIS restriction enzyme. The type IIS restriction recognition sequence becomes double-stranded through hybridization to a helper primer (FIG. 1C).

The additional enzyme acting sequence may comprise digestion sites for RNase H activity when the target is RNA. In fact, the RNase H digestion sites form part of target complementary region of the guide oligonucleotide, because the RNA strand in the RNA/DNA hybrid formed by hybridization between the target RNA and guide oligonucleotide is subjected to RNase H cleavage. The target RNA sequence on RNA/DNA duplex can be digested by RNase H at various non-specific sites. In one embodiment, a part of the target complementary region (preferable the 3′ part sequence) may be made by RNA. The hybridization between target RNA sequence and the target complementary region of guide oligonucleotide forms a part with RNA/DNA hybrid and a part with RNA/RNA hybrid. The target RNA on the RNA/RNA hybrid is resistant to RNase H cleavage therefore the target RNA is not completely digested away with RNase H. This approach leaves a part of RNA sequence intact, so that the 3′ end of the digested RNA can be extended by a DNA polymerase.

7. Helper Primer

In some embodiments, the guide oligonucleotide may comprise additional enzyme acting sequence which supports digestion of target sequence strand hybridized to the target complementary region of the guide oligonucleotide. The additional enzyme acting sequence may comprise restriction site, which may further comprise type IIS restriction site or nicking restriction site. The type IIS restriction site or nicking restriction site may comprise double-stranded restriction enzyme recognition sequence. The double-stranded restriction enzyme recognition sequence is formed through hybridization of guide oligonucleotide and helper primer.

The helper primer comprises at least one portion complementary or substantially complementary to a part of the guide oligonucleotide. The helper primer may comprise sequence complementary to the additional enzyme acting sequence with or without its flanking sequences or complementary to a part of additional enzyme acting sequence of the guide oligonucleotide, whereby a hybridization between the helper primer and guide oligonucleotide makes the additional enzyme acting sequence double-stranded or partially double-stranded. It is preferred the additional acting sequence is type IIS restriction site or restriction nicking site. The helper primer is preferred to hybridize to the recognition sequence of the type IIS restriction site or restriction nicking site forming double-stranded functional recognition sequence. The double-stranded recognition sequence of the type IIS restriction site or restriction nicking site allow the enzyme to digest or nick target sequence strand on a hybrid formed by hybridization between guide oligonucleotide and the target sequence.

The helper primer may further comprise at least one target complementary portion, which hybridizes to a target region that is adjacent or substantially adjacent to the target region complementary to the guide oligonucleotide.

Optionally, the helper primer may also carry a ligand in one or more positions, capable of being captured onto a solid support. A ligand conjugated-helper primer provides a convenient way of separating the target DNA from other molecules present in a sample. Once the ligand conjugated-helper primer—target sequence hybrid is trapped on a solid support via the ligand, the solid support is washed thereby separating the hybrid from all other components in the sample.

Enzymes

For some embodiments of the invention, extension of digested target sequence strand is carried out with a nucleic acid polymerase. “Extension” as the term is used herein is the addition of nucleotides to the 3′ hydroxyl end of a nucleic acid wherein the addition is directed by the nucleic acid sequence of a template. Suitable enzymes for these purposes include, but are not limited to, for example, E. coli DNA polymerase I, Klenow fragment of E. coli DNA polymerase I, T4 DNA polymerase, Vent™ (exonuclease plus) DNA polymerase, Vents (exonuclease minus) DNA polymerase, Deep Vent™ (exonuclease plus) DNA polymerase, Deep Vents (exonuclease minus) DNA polymerase, 9.degree.N.sub.m DNA polymerase (New England BioLabs), T7 DNA polymerase, Taq DNA polymerase, Tfi DNA polymerase (Epicentre Technologies), Tth DNA polymerase, Replitherm™ thermostable DNA polymerase and reverse transcriptase. One or more of these agents may be used in the extension step. The extension step produces a double-stranded nucleic acid having at least a functional first restriction site.

The disclosed method also makes the use of restriction enzymes (also referred to as restriction endonucleases) for cleaving double-stranded nucleic acids. Other nucleic acid cleaving reagents also can be used. Preferred nucleic acid cleaving reagents are those that cleave nucleic acid molecules in a sequence-specific manner. Many restriction enzymes are known and can be used with the disclosed method. Restriction enzymes generally have a recognition sequence and a cleavage site. The restriction enzyme recognition sequences vary in length but require a double-stranded sequence. Restriction enzymes are widely available commercially, and procedures for using them are well known to persons of ordinary skill in the art of molecular biology. The restriction enzyme that cleaves at the first restriction site of guide oligonucleotide when double-stranded is referred to as first restriction enzyme. The restriction enzyme that cleaves at the second restriction site of guide oligonucleotide when double-stranded is referred to as second restriction enzyme.

In some embodiments of the invention, the digested parts with identifier sequence and constant region is ligated to each other by a nucleic acid ligase to produce at lease one joined identifier fragment. Any DNA ligase can be used, T4 DNA ligase is a preferred enzyme.

In one embodiment, partially digestion of the hybridized target RNA at predetermined RNA sequences is carried out with a double-stranded ribonuclease. Such ribonucleases nick or excise ribonucleic acid sequences from double-stranded RNA/DNA hybridized strands. An example of a ribonuclease useful in the practice of this invention is RNase H. RNase H is a RNA specific digestion enzyme which cleaves RNA found in DNA/RNA hybrids in a non-sequence-specific manner. Other ribonucleases and enzymes may be suitable to nick or excise RNA from RNA/DNA strands, such as Exo III and reverse transcriptase.

In another embodiment, single-stranded cDNA is used as target source (FIG. 4). cDNA is formed by reverse transcription using a reverse transcriptase and a biotinylated poly dT primer. Any reverse transcriptase that is suitable to make cDNA from RNA can be used.

Target Polynucleotides

The target polynucleotides (also referred to as nucleic acid) which is analyzed by the subject method can be isolated from any cell or collection of cells. Any source of nucleic acid, in purified or non-purified form, can be utilized as the test sample. For example, the test sample may be a food or agricultural product, or a human or veterinary clinical specimen. Typically, the test sample is a biological fluid such as urine, blood, plasma, serum, sputum or the like. Alternatively the test sample may be a tissue specimen suspected of carrying a nucleic acid of interest The nucleic acid to be detected in the test sample is DNA or RNA, including messenger RNA, from any source, including bacteria, yeast, viruses, and the cells or tissues of higher organisms such as plants or animals.

There are a variety of methods known in the art for isolating RNA from a cellular source, any of which may be used to practice the present method. The Chomczynski method, e.g., isolation of total cellular RNA by the guanidine isothiocyanate (described in U.S. Pat. No. 4,843,155) used in conjunction with, for example, oligo-dT streptavidin beads, is an exemplary mRNA isolation protocol. The RNA, as desirable, can be converted to cDNA by reverse transcriptase, e.g., poly(dT)-primered first strand cDNA synthesis by reverse transcriptase. Likewise, there are a wide range of techniques for isolating genomic DNA which are amenable for use in a variety of embodiments of the subject method.

In many embodiments of the invention, multiple guide oligonucleotides are selected to be used in conjunction with one another, i.e., set of guide oligonucleotides, thereby providing for the simultaneous analysis of multiple polynucleotides when the different oligonucleotides are used in conjunction with one another.

The term “oligonucleotide” or “oligo” as used herein are used broadly to refer to any naturally occurring nucleic acid, or any synthetic analogs thereof, that have the chemical properties required for use in the subject methods, e.g., the ability to sequence specifically hybridize different polynucleotides. Thus, examples of oligonucleotides include DNA, RNA, phosphorthioates PNAs (peptide nucleic acids), phosphoramidates and the like. Method for synthesizing oligonucleotides are well known to those skilled in the art, examples of such synthesis can be found for example in U.S. Pat. Nos. 4,419,732; 4,458,066; 4,500,707; 4,668,777; 4,973,679; 5,278,302; 5,153,319; 5,786,461; 5,773,571; 5,539,082; 5,476,925; and 5,646,260.

The term “ligating” or “joining” as used herein, with respect to oligonucleotides or polynucleotides refers to the covalent attachment of two separate nucleic acids to produce a single larger nucleic acid with a contiguous backbone. Preferred methods of joining are ligase (e.g., T4 DNA ligase) catalyzed reactions. However, non-enzymatic ligation methods may also be employed. Examples of ligation reactions that are non-enzymatic include the non-enzymatic ligation techniques described in U.S. Pat. Nos. 5,780,613 and 5,476,930, which are herein incorporated by reference.

The materials described above can be packaged together in any suitable combination as a kit useful for performing the disclosed method.

Examples of methods of the invention are outlined below.

In one embodiment, two or more sets of guide oligonucleotides are incubated with target RNA or DNA (FIG. 2). Target specific hybridization between guide oligos and target RNA or DNA occurs under optimal hybridization condition. Optionally, following target specific hybridization, biotinylated guide oligos are bound to avidin immobilized on a solid support and undergo stringency washing. If the target is RNA, the target RNA strand on the double-stranded RNA/DNA hybrid on the target complementary region of the guide oligos is partially digested or nicked by RNase H activity. If an additional restriction cleavage or nicking site is located within the target complementary region, the target DNA strand on the double-stranded DNA/DNA hybrid on the target complementary region of the guide oligos is nicked by a restriction enzyme digestion. Alternatively, the single-stranded target sequence 3′ to the target region hybridized to guide oligonucleotide is trimmed with an exonuclease activity which preferably is the 3′-5′ exonuclease activity associated with many nucleic acid polymerases. The 3′ end of the digested, nicked or trimmed target sequence strand is extended by a nucleic acid polymerase using the guide oligonucleotides as templates, whereby the downstream sequences 5′ to the target complementary region of the guide oligonucleotide including the first restriction site become double-stranded. The resulting guide oligonucleotide intermediates are bound (if not captured in any of above steps) to avidin immobilized on a solid support and undergo stringency washing. The parts with identifier sequence and constant region of guide oligos are released from solid support by first restriction enzyme digestion on the first restriction site. The released digested parts of guide oligos can be detected directly by various methods such as mass spectrometry, electrophoresis and microarray. Alternatively, the digested identifier parts from different sets of guide oligos are randomly joined together by ligation using a DNA ligase. The joined parts are amplified by PCR or other amplification method using primers complementary or identical to constant regions of guide oligos. After amplification, the amplicons are digested by first and second restriction enzymes to release individual identifier sequences which then can be detected with various methods for example mass spectrometry. Preferably, the amplicons are digested by second restriction enzyme to release jointed identified fragments, which then can be concatenated by ligation. The concatemers can be cloned and sequenced, therefore the identifier's identities and quantity can be determined.

In another embodiment (FIG. 3), a method is provided for analyzing complex polynucleotides using guide oligonucleotides having their first restriction sites and identifier sequences forming part of target complementary regions. Two sets of guide oligonucleotides are incubated with target RNA or DNA. First set of guide oligonucleotides contains guide oligonucleotides having functional regions in an order from 5′ end to 3′ end as constant region, second restriction site, identifier sequence, first restriction site and target complementary region. Second set of guide oligonucleotides contains guide oligonucleotides having functional regions in an order from 5′ end to 3′ end as target complementary region, first restriction site, identifier sequence, second restriction site and constant region. The two sets of guide oligonucleotides may comprise the same first restriction site, the same second restriction site, but different constant region sequences. Target specific hybridization between guide oligonucleotides and target RNA or DNA occurs under optimal hybridization condition. Optionally, following target specific hybridization, biotinylated guide oligos are bound to avidin immobilized on a solid support and undergo stringency washing. The double-stranded RNA/DNA or DNA/DNA hybrids are digested by first restriction enzyme at first restriction sites. This digestion releases the digested fragments with identifier sequence and constant region from solid support. The released digested identifier fragments can be detected directly by various methods such as mass spectrometry, electrophoresis and microarray. Alternatively, the digested identifier parts from different sets of guide oligos are randomly joined together by ligation using a DNA ligase. The joined parts are amplified by PCR or other amplification method using primers complementary or identical to constant regions of guide oligos. After amplification, the amplicons are digested by first and second restriction enzymes to release individual identifier sequences which then can be detected with various methods for example mass spectrometry. Preferably, the amplicons are digested by second restriction enzyme to release jointed identified fragments, which then can be concatenated by ligation. The concatemers can be cloned and sequenced, therefore the identifier's identities and quantity can be determined.

In still another embodiment (FIG. 4), a method is provided for analyzing biotinilated cDNA. cDNA is generated by reverse transcription of mRNA using a reverse transcriptase and a biotinylated poly dT primer. The cDNA is divided into two pools and each hybridizes to a set of guide oligonucleotide. The two sets of guide oligonucleotides have different constant regions in different orientations. The cDNA is immobilized on a solid support by binding to avidin. The hybrids of cDNA and guide oligonucleotides are then digested with a first restriction endonuclease. The digested parts with identifier sequence and constant region of guide oligos are isolated, and the isolated parts from different pools are mixed and randomly joined together by ligation using a DNA ligase. The joined parts are amplified by PCR or other amplification method using primers complementary or identical to constant regions of guide oligos. After amplification, the amplicons are digested with first and second restriction enzymes to release individual identifier sequences which then can be detected with various methods for example mass spectrometry. Preferably, the amplicons are digested with second restriction enzyme to release jointed identified fragments, which then can be concatenated by ligation. The concatemers can be cloned and sequenced, therefore the identifier's identities and quantity can be determined.

The major steps of method are described as follows:

A. Target Specific Hybridization

A guide oligonucleotide or a set of guide oligonucleotides or more than one set of guide oligonucleotides are incubated with a sample containing DNA, RNA, or both, under suitable hybridization conditions, so that double-stranded DNA/DNA or RNA/DNA or RNA/RNA hybrid on the target complementary regions of the guide oligonucleotides are formed.

Denaturing a nucleic acid sample containing target polynucleotides may be necessary to carry out the assay of the present invention in cases where the target polynucleotide is found in a double-stranded form or has a propensity to maintain a rigid structure. Denaturing is a step producing a single stranded nucleic acid and can be accomplished by several methods well-known in the art (Sambrook et al. (1989) in “Molecular Cloning: A Laboratory Manual,” Cold Spring Harbor Press, Plainview, N.Y.). One preferred method for denaturation may be heat, for example 90-100.degree. C., for about 2-20 minutes.

Alternatively, a base may be used as a denaturant when the nucleic acid is a DNA. Many known basic solutions are useful for denaturation, which are well-known in the art. One preferred method uses a base, such as NaOH, for example, at a concentration of 0.1 to 2.0 N NaOH at a temperature of 20-100.degree. C., which is incubated for 5-120 minutes. Treatment with a base, such as sodium hydroxide not only reduces the viscosity of the sample, which in itself increases the kinetics of subsequent enzymatic reactions, but also aids in homogenizing the sample and reducing background by destroying any existing DNA-RNA or RNA-RNA hybrids in the sample.

The target nucleic acid molecules are hybridized to the target complementary regions of guide oligonucleotides. Hybridization is conducted under standard hybridization conditions well known to those skilled in the art. Reaction conditions for hybridization of an oligonucleotide to a nucleic acid sequence vary from oligonucleotide to oligonucleotide, depending on factors such as the length of target complementary region of a guide oligonucleotide, the number of G and C nucleotides, and the composition of the buffer utilized in the hybridization reaction. Moderately stringent hybridization conditions are generally understood by those skilled in the art. Higher specificity is generally achieved by employing incubation conditions having higher temperatures, in other words more stringent conditions. Chapter 11 of the well-known laboratory manual of Sambrook et al., MOLECULAR CLONING: A LABORATORY MANUAL, second edition, Cold Spring Harbor Laboratory Press, New York (1990) (which is incorporated by reference herein), describes hybridization conditions for oligonucleotide probes and primers in great detail, including a description of the factors involved and the level of stringency necessary to guarantee hybridization with specificity.

Hybridization is typically performed in a buffered aqueous solution, for which the conditions of temperature, salts concentration, and pH are selected to provide sufficient stringency such that the guide oligonucleotide will hybridize specifically to the target nucleic acid sequence but not any other sequence.

If the guide oligonucleotide comprises capture moiety, for example biotin, on its 3′ or 5′ end, the hybridization between a set or several sets of such guide oligonucleotides and target polynucleotides can be performed in a single tube. When the target polynucleotides are cDNA, wherein the oligo dT primer for cDNA synthesis is biotinylated, and the guide oligonucleotides in a set or sets have their first restriction sites and identifier sequences located within their target complementary regions, the cDNA is separated into two pools, each of which is hybridized to different set of the guide oligonucleotides. The guide oligonucleotides in the different set have different order of functional regions. For example, in one set the functional regions of the guide oligonucleotides have the order as from 5′ end to 3′ end: constant region, second restriction site, identifier sequence, first restriction site and target complementary region 3′; whereas in another set the functional regions of the guide oligonucleotides have the order as from 5′ end to 3′ end: target complementary region, first restriction site, identifier sequence, second restriction site, and constant region (FIG. 4).

B. Forming Double-Stranded or Partially Double-Stranded Guide Oligonucleotide Intermediates Including Double-Stranded First Restriction Sites

If the first restriction sites and identifier sequences are part of the target complementary regions of the guide oligonucleotides, the step (B) of forming double-stranded or partially double-stranded guide oligonucleotides intermediates including the first restriction sites is completed after step (A) of target specific hybridization (FIGS. 3 and 4).

If the targets are RNA, the step (B) of forming double-stranded or partially double-stranded guide oligonucleotide intermediates including the first restriction sites may comprise: digesting the target RNA strand of RNA/DNA hybrid by a nuclease, extending the digested strand on guide oligonucleotide templates by a nuclei acid polymerase, whereby the downstream sequences 5′ to the target complementary region of the guide oligonucleotide including the first restriction site become double-stranded (FIG. 2). The nuclease can be RNase H. RNase H is a RNA specific digestion enzyme which cleaves RNA found in DNA/RNA hybrids in a non-sequence-specific manner. To prevent complete digestion of RNA strand in the RNA/DNA hybrid, a portion of target complementary region of the guide oligonucleotide may be made by RNA, thus RNA/RNA hybrid is resistant to cleavage by RNase H

If the guide oligonucleotides comprise additional restriction sites, the step (B) of forming double-stranded or partially double-stranded guide oligonucleotide intermediates including the first restriction sites may comprise: digesting target sequence strand by the restriction enzyme on restriction digestion sites of the additional restriction sites, extending the digested strand on guide oligonucleotide templates by a nucleic acid polymerase, whereby the downstream sequences 5′ to the target complementary region of the guide oligonucleotide including the first restriction site become double-stranded.

If the target complementary regions of the guide oligonucleotides hybridize to free 3′ ends of the target sequences, the step (B) of forming double-stranded or partially double-stranded guide oligonucleotide intermediates including the first restriction sites may comprise: extending said free 3′ ends of the target sequence(s) by a nucleic acid polymerase using said guide oligonucleotides as templates, whereby the downstream sequences 5′ to the target complementary region of the guide oligonucleotide including the first restriction site become double-stranded.

In some embodiments, the step (B) of forming double-stranded or partially double-stranded guide oligonucleotide intermediates including the first restriction sites may comprise: trimming single-stranded target sequence 3′ to the target region hybridized to guide oligonucleotide with an exonuclease activity, extending 3′ ends of the trimmed target sequences by a nucleic acid polymerase using said guide oligonucleotides as templates, whereby the downstream sequences 5′ to the target complementary region of the guide oligonucleotide including the first restriction site become double-stranded. The guide oligonucleotides in these embodiments may comprise at least one modified nucleotide or modified phosphodiester linkage in at least an ultimate 3′ end position to resist exonuclease activity

The trimming step of the present invention may be carried out by various means. The most common method of trimming back 3′ ends utilizes the enzymatic activity of exonucleases. In particular, specific directional exonucleases facilitate a 3′-5′ trimming back of the target DNA-guide oligonucleotide hybrid. Such exonucleases are known within the art and include, but are not limited to, exonuclease I, exonuclease III and exonuclease VII. Preferred, however, is the 3′-5′ exonuclease activity associated with many nucleic acid polymerases. Using such nucleic acid polymerases reduces the number of enzymes required in the reaction and provides the appropriate activity to trim back the free 3′ flanking ends of the target DNA.

After the step (A) or step (B) the method may further comprise: capturing target polynucleotides or guide oligonucleotides or helper primers on solid supports through the end labels, and stringency washing. The 3′ or 5′ end of guide oligonucleotide may be labeled by a capture moiety, for example biotin (FIG. 2 and FIG. 3). Alternatively, the target polynucleotide may be labeled by a capture moiety, for example, a cDNA from mRNA is formed using a biotinylated poly dT primer (FIG. 4). After target specific hybridization or after forming functional first restriction site, the biotin labeled oligonucleotide or polynecleotide are bound to streptavidin on a solid support, for example the beads. A stringency washing may be carried out to remove any unspecific hybridized oligonucleotide or polynucleotide.

C. Digesting the Double-Stranded or Partially Double-Stranded Guide Oligonucleotides with First Restriction Enzyme on the First Restriction Site

Once double-stranded functional first restriction site is formed, the first restriction enzyme acts on and cleaves the double-stranded or partially double-stranded guide oligonucleotides at the first restriction site.

After digesting the double-stranded or partially double-stranded guide oligonucleotides with first restriction enzyme on the first restriction site, the method may further comprises: isolating the digested parts containing identifier sequences and constant regions, which may be attached on the solid support or in supernatant. For example, streptavidin beads are used to isolate the digested part when the oligo dT primer for cDNA synthesis is biotinylated or the guide oligonucleotides are biotinylated. Those of skill in the art will know other similar capture systems (e.g., biotin/streptavidin, digoxigenin/anti-digoxigenin) for isolation of the digested part as described herein.

D. Analyzing the Digested Parts Containing Identifier Sequences and Constant Regions

In one embodiment, the released digested parts of guide oligonucleotides containing constant region and identifier sequence can be detected directly by various methods such as mass spectrometry, electrophoresis and microarray.

In a preferred embodiment of the invention, the isolated digested parts with constant region and identifier sequence can be joined together by DNA ligation. The isolated digested parts may be from one pool of above reaction, or from different pools of above reactions. The joined identifier fragments may be from one set of guide oligonucleotides, or preferably the joined identifier fragments may be from two different sets of guide oligonucleotides. The method of the invention does not require, but preferably comprises amplifying the jointed identifier fragments after ligation. The constant region of guide oligonucleotide comprises sequence for hybridization of an amplification primer. It is preferred that the ligation of identifier fragments is carried out between different sets of guide oligonucleotides with different constant region sequences linked to identifier sequences. In case of analyzing gene expression, each identifier represents at least one gene. The presence of an identifier sequence within the joined fragment is indicative of expression of a gene having a sequence corresponding to a guide oligonucleotide.

The jointed identifier fragments can be amplified by utilizing primers which are complementary or identical to constant regions of guide oligonucleotides. Preferably, the amplification is performed by standard polymerase chain reaction (PCR)methods as described (U.S. Pat. No. 4,683,195). Alternatively, the joined identifier fragments can be amplified by cloning in procaryotic-compatible vectors or by other amplification methods known to those of skill in the art.

The term “primer” as used herein refers to an oligonucleotide, whether occurring naturally or produced synthetically, which is capable of acting as a point of initiation of synthesis when placed under conditions in which synthesis of primer extension product which is complementary to a nucleic acid strand is induced, i.e., in the presence of nucleotides and an agent for polymerization such as DNA polymerase and at a suitable temperature and pH. The primer is preferably single-stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and source of primer.

The amplified jointed fragments then can be analyzed using various detection methods, such as directly DNA sequencing the amplified products. The analysis of joined identifier fragments, formed prior to any amplification step, provides a means to eliminate potential distortions introduced by amplification, e.g., PCR. Alternatively, analyzing the amplified jointed fragments may comprise: digesting the amplified jointed fragments with first and second restriction enzymes at the first and second restriction sites to release individual identifier sequences, detecting and quantifying the identifier sequences by a detection method, such as mass spectrometry, electrophoresis or microarray.

It is preferred that analyzing the amplified jointed fragments may comprise: digesting the amplified jointed fragments with second restriction enzymes to release joined identifier sequences, ligating the joined identifier sequences to produce concatemers, determining the nucleotide sequence of identifier sequences in the concatemers. It is preferred that determining the nucleotide sequence of identifier sequences in the concatemers comprises cloning, sequencing and counting the numbers of identifier sequences. The concatemer may be isolated, preferable as 300 bp to 3 kb fragments, and ligated into a cloning vector to produce a library. The identifier sequence present in a particular clone can be sequenced by standard methods.

Among the standard procedures for cloning the joined identifier fragments or concatemers of the invention is insertion of the fragments into vectors such as plasmids or phage. The joined identifier fragments or concatemers of the joined identifier fragments produced by the method described herein are cloned into recombinant vectors for further analysis, e.g., sequence analysis, plaque/plasmid hybridization, by methods known to those of skill in the art.

The invention also includes kits for performing one or more of the different methods for analyzing polynucleotide population described herein. Kits generally contain two or more reagents necessary to perform the subject methods. The reagents may be supplied in pre-measured amount for individual assays so as to increase reproducibility.

In one embodiment, the subject kits comprise guide oligonucleotides and primers. The kits of the invention may also include one or more additional reagents required for various embodiments of the subject methods. Such additional reagents include, but are not limited to: restriction enzymes, DNA polymerases, buffers, nucleotides, and the like.

### EXAMPLES

1 μg mRNA from mouse spleen was converted to first strand cDNA using a BRL cDNA synthesis kit following the manufacturer's protocol, using the primer biotin-5′poly(T)19-3′. After the first strand cDNA synthesis, the mRNA strand was digested by RNase H. The first strand cDNA was divided into two pools, each of which was incubated with a set of guide oligonucleotides under standard hybridization condition. The first set contains the following guide oligos:

| | |
|---|---|
| [symbol] GAATTCGAGAACAAAGGAT | (J00443) |
| CCACACCCC 3′ | |
| [symbol] GAATTCCATCTGTATCGAG | (BC042693) |
| ATCTGACTCTGTCTTC 3′ | |
| [symbol] GAATTCGAAGCACAGAATG | (BC036266) |
| ATCAGGCCTTTAGAGC 3′ | |
| [symbol] GAATTCCTGCAGGCGGAGA | (BC044785) |
| TCTTCCAGGCCCG 3′ | |
| [symbol] GAATTCGAAGGGGTGAAGA | (BC002116) |
| TCTCCTTGGAGTC 3′ | |

The second set contains the following guide oligos:

| | |
|---|---|
| 5′ AAACAAACGGTGGATCAGAATAGCCACGAATTC [symbol] | (BC023197) |
| 5′ GATAGGCTGAGATCGAGAAATTCGATAAGAATTC [symbol] | (NM_021278) |
| 5′ GAACTGGAAGATCTTCGAGAGCTGGAATTC [symbol] | (NM_010545) |
| 5′ CCCGAGGGAGAGATCACGGACTACAGAATTC [symbol] | (NM_020583) |
| 5′ CTCCTGGCCATGATCATAGCCCCCATGAATTC [symbol] | (NM_019444) |

Constant regions are marked in bold italic letters; first and second restriction sites are underlined.

After hybridization, the cDNA was immobilized on a solid support by binding to magnetic streptavidin beads (Dynal). After extensive washing to remove unhybridized guide oligonucleotides, the hybrids of cDNA and guide oligonucleotides were then digested with the first restriction endonuclease Dpn II. The digestion reactions in this step and in other digestion steps were performed at 25-27 degree C. to keep the oligos annealing to the cDNA. The digested parts with identifier sequence and constant region of guide oligos were isolated, which were performed at 4-18 degree C. In the first pool, the digested parts with identifier sequence and constant region of guide oligos were bound to the beads, whereas in the second pool, the digested parts with identifier sequence and constant region of guide oligos were in the supernatant. The isolated parts from two pools were mixed and randomly joined together by ligation using T4 DNA ligase. The joined parts were amplified for 30 cycles by PCR using primers 5′-GTAAAACGACGGCCAGTG-3′ and 5′-GGAAACAGCTATGACCATG-3′. The PCR reaction was then analyzed by polyacrylamide gel electrophoresis and the desired product excised. The excised amplicons were digested with second restriction enzyme EcoR I and the band containing the joined identifier fragments was excised and self-ligated. After ligation, the concatenated joined identifier fragments were separated by polyacylamide gel electrophoresis and products greater than 300 bp were excised. These products were cloned into the EcoR I site of pBluescript (Stratagene). Colonies were screened for inserts by PCR using T7 and T3 sequences outside the cloning site as primers. Clones containing at least 20 joined identifier fragments were identified by PCR amplification and sequenced.

50 clones were sequenced which contained 828 identifier sequences. The following table shows analysis of the 828 identifier sequences. All ten transcripts were derived from genes of known function in mouse spleen and their prevalence was consistent with previous analyses of spleen RNA.

Identifier sequence and first

| | | |
|---|---|---|
| restriction site | Number | Percent |

| | | |
|---|---|---|
| GAGAACAAAGGATC (J00443) | 128 | 15.5 |
| CATCTGTATCGAGATC (BC042693) | 89 | 10.7 |
| GAAGCACAGAATGATC (BC036266) | 59 | 7.1 |
| CTGCAGGCGGAGATC (BC044785) | 40 | 4.8 |
| GAAGGGGTGAAGATC (BC002116) | 39 | 4.7 |
| GATCAGAATAGCCAC (BC023197) | 45 | 5.4 |
| GATCGAGAAATTCGATAA (NM_021278) | 285 | 34.4 |
| GATCTTCGAGAGCTG (NM_010545) | 98 | 11.8 |
| GATCACGGACTACA (NM_020583) | 20 | 2.4 |
| GATCATAGCCCCCAT (NM_019444) | 25 | 3.0 |

Incorporation By Reference

All publications, patent applications, and patents referenced in the specification are herein incorporated by reference to the same extent as if each individual publication or patent application was specifically and individually indicated to be incorporated by reference.

Equivalents

All publications, patent applications, and patents mentioned in this specification are indicative of the level of skill of those skilled in the art to which this invention pertains. Although only a few embodiments have been described in detail above, those having ordinary skill in the molecular biology art will clearly understand that many modifications are possible in the preferred embodiment without departing from the teachings thereof. All such modifications are intended to be encompassed within the following claims. The foregoing written specification is considered to be sufficient to enable skilled in the art to which this invention pertains to practice the invention. Indeed, various modifications of the above-described modes for carrying out the invention which are apparent to those skilled in the field of molecular biology or related fields are intended to be within the scope of the following claims

What is claimed is:

1. A guide oligonucleotide comprising single-stranded or partially double-stranded nucleic acid, which comprises: target complementary region, constant region, identifier sequence, at least one restriction site.

2. The guide oligonucleotide of claim 1, wherein said at least one restriction site comprises first and second restriction sites which are different, wherein said second restriction site is adjacent to said constant region.

3. The guide oligonucleotide of claim 1, wherein said identifier sequence is specific for each said guide oligonucleotide and is located between the first and second restriction sites.

4. The guide oligonucleotide of claim 1, wherein said constant region is located at the most 3′ or 5′ end of said guide oligonucleotide, wherein said constant region comprises sequence complementary or identical to an amplification primer sequence.

5. The guide oligonucleotide of claim 1 further comprising 5′ or 3′ end label.

6. The guide oligonucleotide of claim 5, wherein said end label comprises biotin.

7. The guide oligonucleotide of claim 1, wherein said identifier sequence and first restriction site are part of target complementary region.

8. The guide oligonucleotide of claim 1, wherein said identifier sequence and first restriction site are not part of target complementary region.

9. The guide oligonucleotide of claim 1 further comprising additional enzyme acting sequence which supports digestion of target sequence strand hybridized to said target complementary region of said guide oligonucleotide.

10. The guide oligonucleotide of claim 9, wherein said additional enzyme acting sequence comprises restriction site.

11. The guide oligonucleotide of claim 10, wherein said restriction site comprises type IIS restriction site or nicking restriction site.

12. The guide oligonucleotide of claim 11, wherein said type IIS restriction site or nicking restriction site comprise double-stranded restriction enzyme recognition sequence.

13. The guide oligonucleotide of claim 10, wherein nucleotides of the cleavage site of said restriction site on the target complementary region are modified, whereby the modified nucleotides are resistant to cleavage.

14. The guide oligonucleotide of claim 13, wherein said modified nucleotides comprise phosphorothioate linkages.

15. The guide oligonucleotide of claim 9, wherein said additional enzyme acting sequence comprises RNase H digestion sites when the target is RNA.

16. The guide oligonucleotide of claim 15, wherein the target complementary region of said guide oligonucleotide comprises chimeric RNA and DNA.

17. A set of guide oligonucleotides comprising multiple guide oligonucleotides each having a target specific target complementary region, a guide oligonucleotides specific identifier sequence, the same first restriction site, the same second restriction site, and the same constant region sequence.

18. A method of analyzing polynucleotides in a sample, said method comprising steps of:
(a) hybridizing guide oligonucleotides or a set of guide oligonucleotides or more than one set of guide oligonucleotides in accordance with any one of the preceding claims to target polynucleotides, whereby target complementary regions of said guide oligonucleotides become double-stranded if the target sequences are present in the sample;
(b) forming double-stranded or partially double-stranded guide oligonucleotide intermediates including double-stranded first restriction sites;
(c) digesting said double-stranded or partially double-stranded guide oligonucleotides intermediates with first restriction enzyme at the first restriction site; and
(d) analyzing the digested parts containing identifier sequences.

19. The method of claim 18, wherein the first restriction site and identifier sequence are part of the target complementary region of the guide oligonucleotide, and said step (b) is completed after said step (a).

20. The method of claim 18, wherein the target polynucleotides are RNA, and said step (b) of forming double-stranded or partially double-stranded guide oligonucleotide intermediates comprises: digesting the target RNA strand of RNA/DNA hybrid by a nuclease, extending the 3′ end of the digested strand on guide oligonucleotide templates by a nucleic acid polymerase, whereby the downstream sequences 5′ to the target complementary region of the guide oligonucleotide including the first restriction site become double-stranded.

21. The method of claim 20, wherein said nuclease is RNase H.

22. The method of claim 18, wherein the guide oligonucleotide comprises additional restriction site, and said step (b) of forming double-stranded or partially double-stranded guide oligonucleotide intermediates comprises: digesting target sequence strand at the restriction digestion site of said additional restriction site by a restriction enzyme, extending the 3′ end of the digested strand on guide oligonucleotide templates by a nucleic acid polymerase, whereby the downstream sequences 5′ to the target complementary region of the guide oligonucleotide including the first restriction site become double-stranded.

23. The method of claim 18, wherein the target complementary regions of said guide oligonucleotides hybridize to free 3′ ends of the target sequences, and said step (b) of forming double-stranded or partially double-stranded guide oligonucleotide intermediates comprises: extending said free 3′ ends of the target sequences by a nucleic acid polymerase using said guide oligonucleotides as templates, whereby the downstream sequences 5′ to the target complementary region of the guide oligonucleotide including the first restriction site become double-stranded.

24. The method of claim 18, wherein said step (b) of forming double-stranded or partially double-stranded guide oligonucleotide intermediates comprises: trimming single-stranded target sequence 3′ to the target region hybridized to the guide oligonucleotide with an exonuclease activity, extending 3′ ends of the trimmed target sequences by a nucleic acid polymerase using said guide oligonucleotides as templates, whereby the downstream sequences 5′ to the target complementary region of the guide oligonucleotide including the first restriction site become double-stranded.

25. The method of claim 24, wherein said guide oligonucleotide comprises at least one modified nucleotide or modified phosphodiester linkage in at least an ultimate 3′ end position to resist exonuclease activity.

26. The method of claim 18 further comprising: after said step (a) or after step (b) capturing said polynucleotides or said oligonucleotide on a solid support through the end labels, and stringency washing.

27. The method of claim 18 further comprising: after said step (c) isolating the digested parts containing identifier sequences and constant regions, wherein said digested parts are attached on solid support or in supernatant.

28. The method of claim 18, wherein said step (d) of analyzing the digested parts containing identifier sequences comprises: detecting said digested parts by mass spectrometry, electrophoresis or microarray.

29. The method of claim 18, wherein said step (d) of analyzing the digested parts containing identifier sequences comprises: ligating said digested parts to each other by a nucleic acid ligase to produce at lease one joined identifier fragment, amplifying joined identifier fragments using primers that are complementary or identical to constant regions of the guide oligonucleotides, analyzing the amplified products.

30. The method of claim 29, wherein said analyzing the amplified products comprises determining the nucleotide sequences of said amplified products.

31. The method of claim 29, wherein said analyzing the amplified products comprises: digesting said amplified products with first and second restriction enzymes to release individual identifier sequences, detecting and quantifying said identifier sequences by a detection method.

32. The method of claim 31, wherein said detection method comprises mass spectrometry, electrophoresis or microarray.

33. The method of claim 29, wherein said analyzing the amplified products comprises: digesting said amplified products with second restriction enzymes to release joined identifier fragments, ligating said joined identifier fragments to produce concatemers, determining the nucleotide sequence of identifier sequences in said concatemers.

34. The method of claim 33, wherein said determining the nucleotide sequence of identifier sequences in said concatemers comprises: cloning, sequencing and counting the numbers of identifier sequences.

35. The method according to claim 18 wherein said polynucleotide is RNA, cDNA or genomic DNA.
